(19) United States
(12) Patent Application Publication
(10) Pub. No.: US 2006/0078605 A1
(43) Pub. Date: Apr. 13, 2006

(54) Pharmaceutical composition of small-sized liposomes and method of preparation

(76) Inventor: Carlos Alberto Genaro Mammarella, Buenos Aires (AR)

Correspondence Address: The H T Than Law Group;Waterfront Center, Suite 560, 1010 Wisconsin Avenue N W, Washington, DC, 20007, US

(21) Appl. No.: 10/526,180

(22) PCT Filed: Aug. 28, 2003
(86) PCT No.: PCT/BR03/00123

§ 371 Date: Nov. 23, 2005

(30) Foreign Application Priority Data
Aug. 29, 2002 (AR) P 02 01 03264

(51) Int. Cl. A61K 31/704; A61K 31/337; A61K 9/127

(52) U.S. Cl. 424/450; 514/34; 514/449

(57) ABSTRACT

A pharmaceutical composition of small sized unilamellar liposomes for the supply active principles by injection, with an improved permanency in the blood flow, where the unilamellar membrane contains a mixture of saturated lipids encompassing at least one lysophospholipid in a quantity from about 0.5 mol % to about 6.0 mol % with reference total lipids and the production method. Additionally, liposomes of high encapsulation efficiency of an active principle like doxorubicine are prepared through the adding of a solution of calcium ions.

### FIELD OF THE INVENTION

The present invention refers to novel compositions of small-sized liposomes, with the aim to supply active compounds by injectable route, especially for therapeutical applications, with enhanced permanency in blood. In addition, a preparation method for liposomes with high incorporation efficiency of the active principle within the liposomes, is provided.

### BACKGROUND OF THE INVENTION

Most of the drugs administered by perfusion or through injection, have at least, one of the following drawbacks:

1) they are rapidly eliminated from the circulation, or

2) they have a low therapeutic index (particularly due to a high toxicity, to a high incidence of adverse effects compared to its therapeutic efficiency or to a bad distribution).

In this context, liposomes have been widely used as systems for the controlled and sustained delivery of active principles.

Liposomes are essentially lipidic vesicles, suspended in an aqueous medium, containing an entrapped aqueous volume within them.

Liposomes are essentially completely closed spherical structures formed by double-layer lipid membranes. Liposomes may be unilamellar vesicles (possessing a single membrane bilayer) or multilamellar vesicles (onion-like structures characterized by multiple membrane bilayers, each separated from the next by an aqueous layer). The bilayer is composed of two monolayers of molecules of a particular type, having a hydrophobic (“tail”) region and a hydrophilic (head) region. This type of molecules is called amphipatic. The structure of the membrane bilayer is such that the hydrophobic (non polar) “tails” of the lipid monolayers orient toward the center of the bilayer while the hydrophilic (polar) “heads” orient towards the aqueous phase. The resulting structure is an energetically stable, closed structure able to transport bioactive molecules. Thus, the bioactive molecules trapped in the liposomes may present a better therapeutic index, as well as an improved bio-distribution. The drugs transported by liposomes are gradually delivered to the circulation, lessening the toxic side effects associated with the administration of the free drug.

The liposomes are extensively used for the preparation of pharmaceutical formulations, to supply a variety of active agents of diagnostic and therapeutical value in a selective manner.

Liposomes constituted by different lipids were described in the prior art, for example in U.S. Pat. No. 4,737,323 (1988), U.S. Pat. No. 4,769,250 (1988), U.S. Pat. No. 4,837,028 (1989), U.S. Pat. No. 4,863,739 (1989), U.S. Pat. No. 4,920,016 (1990), U.S. Pat. No. 5,013,556 (1991), U.S. Pat. No. 5,463,066 (1995). At the same time, different methods and variants thereof, are known for the preparation of different types of liposomes, such as the ones described in the following publications: Preparation of liposomes of defined size distribution by extrusion through polycarbonate membranes, by Olson, F., Hunt, C. A., Szoka, F. C, Vail, W. J., Papahadjopoulos, D., Biochem Byophys. Acta, 557, 9-23 (1979), Vesicles of variable size produced by a rapid extrusion procedure, by Mayer, L. D., Hope, M. J. Cullis, P. R. Biochem. Biophyhs. Acta 858, 161-168 (1986), and Effects of soluble concentrations on the entrapment of solutes in phospholipids vesicles prepared by freeze-thaw extrusion, by Chapman, C. J.; Erdahl, E. E.; Taylor, R. W; Pfeiffer, D. R.; Chem. Phys. Lipids, 60, 201-208 (1991). Additional bibliographical information may be found in: N. Berger, A. Sachse, J. Bender, R. Schbert and M. Branddl, Int. J. Pharmaceutics, 223, 55-68 (2001). Some patents, such as for example U.S. Pat. No. 5,043,164, describe liposome compositions containing lysophospholipids. Said patent describes a composition for liposomes containing a phosphatidyl ethanolamine (particularly dioleyl phosphatidyl ethanolamine) and a fatty acid, such as oleic acid. According to the disclosure of the above mentioned patent, with the aim of stabilizing the liposomes, cholesterol (a conventional stabilizing agent) is replaced by an amphypatic substance, such as, lysophospholipids, gangliosides, sulphatides, lyophilic drugs and amphipathic proteins wherein the ratio of lipids: amphypatic substance is from about 10:1 to 1:1 w/w and wherein said amphypatic substance is added a short time after the preparation of the liposomes.

On the other hand, U.S. Pat. No. 5,009,956 describes a method to stabilize liposome membranes using a mixture of a phospholipid and between 20-30 mol % of a lysophospholipid, wherein at least one of them is unsaturated.

As mentioned above, liposomes have been extensively used as systems for the controlled and sustained release of active compounds retained inside the liposomes during a prolonged period of time. In this way, by limitation of the concentration of free drug in the blood flow, the possible toxic effects of drug, are reduced. Nevertheless, a frequent problem of this strategy emerges through the swift elimination of liposomes by the reticular endothelial system (RES) and the low retention of the active principles.

One of the factors contributing to reduce to a minimum the removal of the liposomes by the RES is the liposome preparation of small and uniform size.

Another factor contributing to improve the therapy of the delivery of active compounds, constitutes the possibility of obtaining liposomes with an improved efficiency to load active compounds, thus enhancing the amount of active compound entrapped inside the liposome vesicles.

### SUMMARY OF THE INVENTION

The present invention provides a pharmaceutical composition of small-sized unilamellar liposomes for the delivery of an injectable active compound.

According to the present invention, liposomes of small size are obtained by the addition of limited quantities of a lysophospholipid to the lipid mixture that constitutes the membrane formulation.

Thus, it is an object of the present invention, to provide a pharmaceutical composition of small-sized, sucrose liposomes for a parenteral administration of an active compound, which comprises: (i) liposomes with an average diameter of about 75 nm to about 300 nm, wherein the unilamellar membrane is formed by a mixture of saturated lipids containing a ratio of lysophospholipids of about 0.5 mol % to about 6.0 mol % of the total lipids content, and (ii) an encapsulated therapeutic compound contained in said liposomes.

Preferred concentrations of lysophospholipids are those of about 1.4 mol % and about 2.8 mol % regarding the total lipid content.

It is another object of the invention to provide a method for the preparation of small sized liposomes, wherein said method is controlled through a simple procedure, by the adding of small quantities of a lysophospholipid to the lipidic mixture used in said preparation.

In a particular embodiment of the invention, it is provided a pharmaceutical composition of unilamellar liposomes of small size, for a parenteral administration of a cytotoxic agent, wherein said cytotoxic agent is preferably an anthracyclinic antibiotic such as doxorubicin, epirubicin or daunorubicin. In a preferred embodiment of the invention, doxorubicin is used.

Another outstanding aspect of the present invention is a method for the preparation of a liposome composition aimed to enhance the amount of encapsulated doxorubicine in the liposomic vesicles. Such improvement in the efficiency of doxorubicin incorporation into the liposomes is obtained by adding calcium ions to the doxorubicin solution during the step of loading the liposomes with active principle.

### DETAILED DESCRIPTION OF THE DRAWINGS

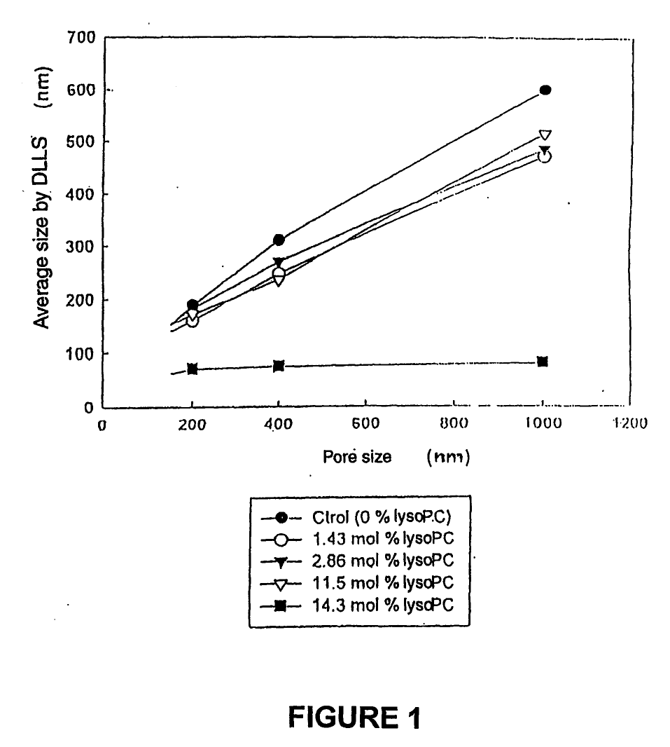

FIG. 1 shows the size distribution curves of liposomes for increasing quantities of lysophospholipids, as a function of the pore size of the extruding polycarbonate membrane.

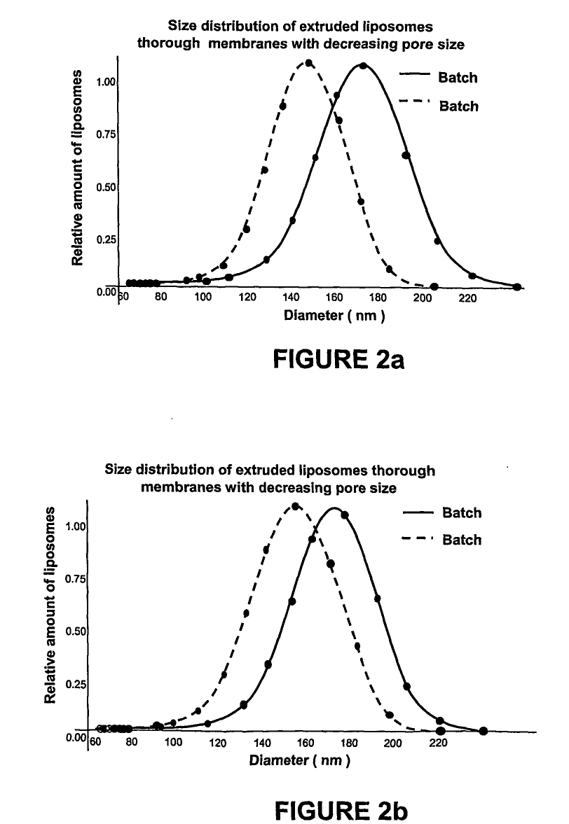

FIGS. 2a y 2b show the size distribution of extruded liposomes through membranes with decreasing pore size (in this case the smaller size pore depicted is 200 nm), with addition of lysophospholipid (batches 06012 and 06013) and without the addition of lysophospholipid (batch 06011).

### DETAILED DESCRIPTION OF THE INVENTION

The liposomes of the present invention are unilamellar liposomes having a single double layer membrane. The bilayer is composed of two monolayers of molecules of a particular type (amphipathic molecules), having a hydrophobic (“tail”) region and a hydrophilic (head) region. The structure of the bilayer membrane is such that the hydrophobic (non polar) “tails” of the lipid monolayers orient toward the center of the bilayer while the hydrophilic (polar) “heads” orient towards the aqueous phase. The resulting structure is an energetically stable, closed structure, able to transport bioactive molecules.

The unilamellar membrane of this invention is formed by a mixture of saturated lipids. According to the present invention, small-sized liposomes are obtained by adding lysophospholipids to the mixture of lipids used for the preparation of liposomal membrane. Preferably, the lysophospholipids are selected from lysophosphatidylcholine, lysophosphatidylinositol, lysophosphatidylserine and lysophosphatidic acid. Lysophosphatidylcholine (Lyso PC)), is obtainable by chemical synthesis or by enzymatic hydrolysis with phospholipase A2. Naturally it is also produced as a degradation product of phosphatidylcholine.

The lipids used for the preparation of the unilamellar membrane are saturated lipids, preferably selected among phosphatidylcholine, cholesterol and phosphatidyl ethanolamine, phosphatidylinositol, phosphatidylglycerol, natural phosphatidylcholine (from soybean and/or eggs) and hydrogenated phosphatidylcholine obtained from different natural sources like soybean or eggs, distearoyl fosfatidylethanolamine derivatized with polyethyleneglycol 2000-O-methylated and/or glycolipids like GM1 or other sialogangliosides, or combinations thereof.

It was found experimental evidence that the addition of increasing, but limited quantities of a lysophospholipid to the mixtures of lipids used in the preparation of liposomes, produce a reduction of the liposomes size, when they are compared to those produced using the same mixture of lipids without the addition of lysophospholipids.

As used herein, liposomes of small size are liposomes presenting an average diameter lower than about 500 nm, preferably an average diameter that ranges from about 75 nm to about 300 nm. Big size liposomes are those having an average diameter of about 500 nm. The average diameter may be determined through conventional, well-known methods, for the skilled in the art. Among such methods, electronic microscopy and dynamic laser light dispersion may be mentioned. (Laser Light Scattering).

According to the present invention, liposomes of small size are obtained by the addition of lysophospholipids to the lipid mixture that will conform the liposomal membrane, preferably with a content of lysophospholipid which varies between about 0.5 mol % and about 6.0 mol % related to the total amount of lipid content. Most preferably, the content of lysophospholipids could be from about 1.4 mol % to about 2.8 mol %, related to the total amount of lipid content.

Sterols may be conveniently added to the mixture of lipids. Particularly, cholesterol could be added. The addition of cholesterol increases the stability of the liposomal vesicles, improving the retention of the active principle.

Liposomes are prepared through generally known techniques. Particularly, for the preparation of liposomes of the present invention, a procedure combining freezing/unfreezing cycles with extrusion through membranes of different pore size is preferred. Most preferably, a combination of a homogenization procedure, carried out with an appropriate homogeneizer, and extrusion through membranes of different pore size could be used.

Preferably, the lipid mixture is dissolved in an organic solvent which is evaporated up to dryness. The lipidic membrane formed is taken up with an aqueous solution, the suspension being subjected to 3 and 6 freezing cycles (from about −20° C. to −45° C.) and unfreezing (up to 50° C.-60° C.). Afterwards, the suspension is extruded through polycarbonate membranes [Preparation of liposomes of defined size distribution by extrusion through polycarbonate membranes, by Olson F., Hunt, C. A., Szoka, F. C., Vail, W. J., Papahadjopoulos, D., Biochem. Biophys. Acta, 557, 9-23 (1979); Vesicles of variable size produced by a rapid extrusion procedure, by Mayer, L. D., Hope, M. J., Cullis, P. R.; Biochem. Biophys. Acta 858, 161-168 (1986)]. In the present invention, extrusion starts with the membrane of biggest pore, e.g. 1000 nm, followed by a membrane of smaller pore (400 nm) and following with membranes of the smallest pore size, until liposomes of the desired size are obtained.

The incorporation of the active agent inside the liposomes is made, according to the present invention, by the method of active loading, after the dialysis of the liposome suspension, by known procedures for the skilled in the art.

The efficiency of loading of an active agent into a liposome also depends on the chemical properties of the compound. Generally, compounds soluble in water or soluble in lipids are of easier incorporation. Compounds soluble in lipids could be easily incorporated into the lipidic bilayer during the formation of the liposome(passive loading). On the other hand, compounds soluble in water interact with the polar head of the phospholipid which is confronted with in the interior of the liposome and therefore the compound is easily sequestered in the inside of the liposome. The amphypatic compounds, such as the anthracyclinic antibiotics are the most difficult to retain inside the liposomes.

In another embodiment of the invention, it was found essential, for the supply of therapeutically effective doses of a variety of cytotoxic agents, to load the liposomes with high concentration of the active principle. For example, for cytotoxic agents such as anthracyclinic antibiotics, particularly anthracyclinic antibiotics such as doxorubicin, epirubicin, daunorubicin, salts thereof and similar compounds, it is desirable to obtain a ratio of encapsulated active principle of about 8.5% w/w to about 11.5% w/w referred to the weight of the lipid content of the liposomes.

A method for the active loading of amphypatic drugs in the liposome is described in U.S. Pat. No. 5,192,542 (Barenolz et al.), which is incorporated herein as a reference. According to such method, liposomes are prepared in the presence of ammonium ions, for example in an ammonium sulfate solution or in the presence of any other ammonium compound solution which is able to dissociate within the liposomes, such as phosphate, carbonate and bicarbonate solutions. After achieving the adequate size, the liposome suspension is treated so as to create a gradient of ammonium ions through the liposomal membrane.

Surprisingly, and according to a particular embodiment of the present invention, it was discovered that when the doxorubicin loading within the liposomes is performed in presence of small concentrations of calcium ions, the efficiency of encapsulation is remarkably enhanced.

Particularly preferred starting calcium ions solutions are calcium chloride solutions at a concentration from about 50 mM to about 200 mM. Other soluble salts of calcium may be used. pH Buffering components, when they are used, shall not include sequestrating calcium substances. Acetic/acetate solutions as well as any other anion solution which do not produce calcium ion precipitation may be used. An amino acid, such as histidine, could also be used. The ratio of liposome solution to calcium chloride solution may be of about 1.5 to 0.05-0.5 (v/v).

Without adhering to a particular theory, it is understood that the presence of calcium ions would allow the elimination of the remaining of ammonium sulfate from the outside of the lipidic vesicles, since in the presence of same, doxorubicin gellifies, and in such a case it would not be available to permeate to the interior of the liposomes. The elimination of the remaining ammonium ions to the immediate exterior zone of liposomes, would free doxorubicin to incorporate inside the liposomes. Therefore, a method to improve the yield of incorporation of the active principle inside the liposomes is achieved, increasing the percentage of encapsulated active principle from of about 20 to about 70%, when compared to the incorporation obtained by a method which does not use calcium ions.

The following specific examples are provided as exemplary of the invention but are not limitative.

### EXAMPLES

Control Preparation

A solution containing 95 mg of hydrogenated soybean phosphatidyl choline, 30 mg of phosphatidyl ethanolamine derivatized with O-methyl-polyethylenglycol-2000 and 30 mg of cholesterol in 15 ml of ethanol anhydrous is prepared.

The mixture is evaporated in a rotatory evaporator up to dryness, trying not to exceed a temperature of 45° C. The formed film is taken up in an ammonium sulfate solution at 45° C. (5 ml of a solution containing about 13.2 mg/l), with stirring at room temperature.

The liposomes obtained in the previous step, are subjected to freezing (−45° C.) and unfreezing (thawing) (50° C.) cycles. At least 6 cycles are performed. Afterwards they are extruded through decreasing pore membranes, starting through a membrane of 1000 nm, afterwards 400 nm and finally through a membrane of 200 nm.

The average size of the liposomes in this preparation was determined by the method of Laser Light Scattering. The result is shown in FIG. 1 with a full circle (0 mol % of lysophospholipid/total lipids).

### Example 1

A solution containing 95 mg of hydrogenated soybean phosphatidylcholine, 1.5 mg of palmitoyl lysophosphatidyl choline, 30 mg of phosphatidyl ethanolamine derivatized with O-methyl polyethileneglycol-2000 and 30 mg of cholesterol in 15 ml of anhydrous ethanol is prepared.

The mixture is evaporated in a rotatory evaporator up to dryness, at a temperature not higher than 45° C. The film formed is taken up in a solution of ammonium sulfate at 45° C. (5 ml of a solution containing 13.20 mg/l) under stirring at room temperature. The liposomes obtained in the previous step are submitted to freezing (−45° C.) and thawing (50° C.) cycles. At least 6 cycles are performed.

Afterwards extrusion through decreasing pores membranes is performed, starting with membranes of 1,000 nm, following with a membrane of smaller pore size (400 nm) and finally through a 200 nm membrane. The average size of liposomes in this preparation is shown in FIG. 1 with an empty circle (1.43 mol % of lysophospholipid/total lipids.)

### Example 2

A solution containing 95 mg of hydrogenated soybean phosphatidylcholine, 3 mg of palmitoyl lysophosphatidyl choline, 30 mg of phosphatidyl ethanolamine derivatized with O-methyl polyethylenglycol-2000 and 30 mg of cholesterol in 15 ml of anhydrous ethanol is prepared.

The mixture is evaporated in a rotatory evaporator until dryness, at a temperature not higher than 45° C. The formed film is taken up in a solution of ammonium sulfate at 45° C. (5 ml of solution containing 13.20 mg/l), under stirring at room temperature.

The liposomes obtained in the previous step are submitted to freezing (−45° C.) and thawing cycles (50° C.). At least 6 cycles are performed.

Afterwards extrusion through decreasing pores membranes is performed, starting with membranes of 1000 nm, following with a membrane of smaller pore size (400 nm) and finally through a 200 nm membrane.

The average size of the liposomes obtained in this preparation is shown in FIG. 1, with a full triangle (2.86 mol % of lysophospholipid/total lipids)

### Example 3

A solution containing 95 mg of hydrogenated soybean phosphatidylcholine, 14 mg of palmitoyl lysophosphatidyl choline, 30 mg of phosphatidyl ethanolamine derivatized with methyl polyethylenglycol-2000 and 30 mg of cholesterol in 15 ml of anhydrous ethanol is prepared.

The mixture is evaporated in a rotatory evaporator up to dryness, trying to perform it at a temperature not higher than 45° C. The formed film is taken up in solution of ammonium sulfate at 45° C. (5 ml of solution containing 13/20 mg/l), with stirring at room temperature.

The liposomes obtained in the previous step are submitted to freezing (−45° C.) and thawing (50° C.) cycles. At least 6 cycles are performed.

Afterwards, extrusion through decreasing pores membranes is performed, starting with membranes of 1,000 nm, following with a membrane of smaller pore size (400 nm) and finally through a 200 nm membrane.

The average size of liposomes in this preparation is shown in FIG. 1 with a void triangle (11.5 mol % of lysophospholipid/total lipids).

### Example 4

A solution containing 95 mg of hydrogenated soybean phosphatidylcholine, 18 mg of palmitoyl lysophosphatidyl choline, 30 mg of phosphatidyl ethanolamine derivatized with O-methyl polyethylenglycol 2000 and 30 mg of cholesterol in 15 ml of anhydrous ethanol is prepared.

The mixture is evaporated in a rotating evaporator up to dryness, at a temperature not higher than 45° C. The film formed is taken up in solution of ammonium sulfate at 45° C. (5 ml of a solution containing 13.20 mg/l), with stirring at room temperature.

The liposomes obtained with the former step are submitted to freezing (−45° C.) and thawing (50° C.) cycles. At least 6 cycles are performed.

Afterwards extrusion through decreasing pores membranes is performed, starting with membranes of 1,000 nm, following with a membrane of smaller pore size (400 nm) and finally through a 200 nm membrane.

The average size of the liposomes in this preparation is shown in FIG. 1 with a full square (14.3 mol % of lysophospholipid/total lipids).

On Table 1 the measured sizes of liposomes containing progressive quantities of lysophospholipids after extrusion through membrane of 400 nm, according to the description of the previous examples and FIG. 1, are shown.

TABLE 1

| | | Size measured of liposomes |
|---|---|---|
| | | containing progressive |
| | Lyso PC content in | quantities of Lyso PC after |
| Example | mol % (Lyso PC/Total | extrusion through membrane |
| No. | lipids) | of 400 nm |
| Control | — | 310 nm |
| 1 | 1.43 | 215 nm |
| 2 | 2.86 | 240 nm |
| 3 | 11.5 | 208 nm |
| 4 | 14.3 | 75 nm |

### Example 5

Two batches of liposomes (06012 and 06013) are prepared according to the procedure described in Example 1. Also a liposome batch is prepared, in accordance to the process described in the item “Control preparation” (Batch 06011). FIGS. 2a and 2b depict the distribution of particle size for batches 06012 and 06013, compared with the distribution of particle size for batch 06011 (Control without lysophospholipid).

A consistency of results is observable between the liposome batches containing lysophospholipid. In both batches (06012 and 06013), a smaller size of particles than the corresponding to the control preparation without lysophospholipid (06011), can be seen.

### Example 6

A liposome suspension obtained as described in Example 1 is dialyzed against a solution of sucrose 10% (w/v) in order to eliminate the ammonium sulfate on the outside of the liposomes.

Afterwards, a solution containing the following composition is prepared: 1.5 volumes of liposomes in suspension, 1 volume of a doxorubicin hydrochloride solution containing 6 mg/ml of said compound in a sucrose solution 10% (w/v) and histidine 0.15% (w/v) and 0.5 ml of a solution of sucrose 10%/histidine 0.15% (w/v) (buffer sucrose/histidine).

The mixture is settled during 15 minutes, and warmed up lightly.

The degree of encapsulated doxorubicin hydrochloride is determined by UV spectrometry (absorbancy at 590 nm). With this purpose, absorbancy determinations on samples of liposomes with encapsulated doxorubicin dilutions in alkaline isotonic medium (free of doxorubicin) and on samples of liposome with encapsulated doxorubicine dilutions in alkaline medium containing detergent (total doxorubicin) are performed. Through the absorbancy data obtained at 590 nm the percentage of encapsulation of 78.7% is calculated. The percentage of free doxorubicin is 21.3%.

### Example 7

A suspension of dialyzed liposomes is prepared as described in Example 5, and afterwards said suspension is incubated, according to the following ratios: 1.5 volumes of liposomes suspension, 0.4 volumes of sucrose/histidine buffer (according to example 5), 0.1 volumes of solution 100 mM of Cl2Ca and 1.0 volume of doxorubicin hydrochloride solution, 6 mg/ml in sucrose/histidine buffer.

The mixture is left setting during 15 minutes, with light warming.

The percentage of doxorubicin incorporated is determined (as depicted in example 5), obtaining a value of 87.9%. The percentage of free doxorubicin is 12.1%. As can be seen, the degree of incorporation is 9.2 points higher than the one obtained without Cl2Ca.

### Example 8

A process according to Example 5 is used. Once incubation is finished, dialysation against a buffer solution of sucrose/histidine during 12 hours is performed.

The percentage of encapsulation of doxorubicin hydrochloride is 91.2%.

### Example 9

A process according to Example 6 is used, and finally it is dialyzed against a buffer solution of sucrose/histidine during 12 hours.

The percentage of encapsulated doxorubicine is 95.53%, e.g. 4.33 points higher than without the adding of calcium chloride. In other words, the percentage of free doxorubicin is 50% less than without the addition of calcium chloride.

While the invention has been described in detail, with specific reference to process realizations and particular procedures, it will be noted that different alterations, modifications and adaptations may be based on the present description and are included in the spirit and scope of the present invention as defined in the following claims.

1. A pharmaceutical composition of small-sized, unilamellar liposomes, for parenteral administration of an active compound, comprising: liposomes with an average diameter from about 75 nm to about 300 nm, wherein the unilamellar membrane contains a mixture of saturated lipids, said unilamellar membrane containing at least one lysophospholipid in an amount of about 0.5 mol % to 6.0 mol % regarding the total lipid content, and a therapeutic compound being encapsulated inside said liposomes.

2. A composition according to claim 1, wherein the lysophospholipid is selected from lysophosphatidylcholine, lysophosphatidylinositol, lysophosphatidylserine and lysophosphatidic acid or combinations thereof.

3. A composition according to claim 1, wherein the saturated lipids are selected from phosphatidylcholine, cholesterol, phosphatidylethanolamine, phosphatidylinositol, phosphatidylglycerol, natural phosphatidylcholine (soy and/or egg), distearoyl phosphatidylethanolamine derivatized with O-methylated polyethyleneglycol 750-5000, dipalmitoyl phosphatidylethanolamine derivatized with O-methylated polyethyleneglycol 750-5000 or combinations thereof.

4. A composition according to claim 3, wherein distearoyl phosphatidylethanolamine is derivatized with O-Methyl-polyethylene-glycol 2000.

5. A composition according to claim 3, wherein dipalmitoyl phosphatidyl ethanolamine is derivatized with O-methyl-polyethylene-glycol

6. A composition according to claim 1, wherein the active principle is a cytotoxic agent.

7. A composition according to claim 6, wherein the cytotoxic agent is selected from anthracyclinic antibiotics, taxanes and platinum salts.

8. A composition according to claim 7, wherein the anthracyclinic antibiotic is selected from the group consisting in doxorubicin, epirubicin and daunorubicin and pharmaceutically acceptable salts thereof.

9. A pharmaceutical composition of unilamellar, small sized liposomes for parenteral administration of an active compound, according to claim 1, comprising: liposomes with an average diameter from about 75 nm to about 300 nm, wherein said unilamellar membrane contains a mixture of saturated lipids, comprising at least one lysophospholipid in an amount of about 0.5 mol % to about 6.0 mol % related to the total lipid content, and encapsulated doxorubicin inside said liposomes in a ratio of about 8.5% by weight to about 11.5% by weight related to the total weight of lipids in the liposomes.

10. A method of preparing a composition according to claim 1, comprising the steps of: forming liposomes from a solution containing saturated lipids and at least a lysophospholipid in an amount of about 0.5 mol % and 6.0 mol % regarding the total lipid content, and evaporation to dryness; taking the film up in aqueous solution; submitting the foregoing solution to freezing and thawing cycles extruding through membranes of decreasing pore up to a membrane of 50 nm pore, obtaining liposomes with an average diameter of about 75 nm to about 300 nm, dialyzing the liposome suspension, and mixing the dialyzed liposome suspension with a solution of the active compound.

11. A method according to claim 10, wherein the lysophospholipid is selected among lysophosphatidylcholine, lysophosphatidylinositol, lysophosphatidylserine and lysophosphatidic acid.

12. A method of preparing a composition of claim 9, comprising the steps of: forming liposomes from a solution containing saturated lipids and at least a lysophospholipid in an amount of about 0.5 mol % to 6.0 mol % regarding the total lipid content, and evaporating to dryness; taking the film up in a solution of an ammonium salt; submitting the foregoing solution to freezing and thawing cycles extruding through membranes of decreasing pore up to a membrane of 50 nm pore, obtaining liposomes with an average diameter of about 75 nm to about 300 nm; dialyzing the liposome suspension against an aqueous solution without ammonium ions; mixing the dialyzed liposome suspension with a solution of about 50 mM to about 200 mM of a soluble calcium salt and a solution of doxorubicin at a concentration of about 2 to about 30 mg/ml, obtaining a percentage of more than 80% of encapsulation of doxorubicin.

13. A method according to claim 12, wherein the calcium salt is calcium chloride.

14. A method according to claim 12, wherein the volume ratio of calcium chloride solution to doxorubicin solution is of 1:10 (v:v).

15. A method according to claim 12, wherein the percentage of encapsulated doxorubicin increases between 20 to 70% in the presence of calcium chloride, compared to a method which does not use calcium chloride.

16. A method according to claim 13, wherein the volume ratio of calcium chloride solution to doxorubicin solution is of 1:10 (v:v).
